(19) United States
(12) Patent Application Publication
(10) Pub. No.: US 2004/0171076 A1
(43) Pub. Date: Sep. 2, 2004

(54) Detectable micro to nano sized structures, methods of manufacture and use

(76) Inventors: Matthew J. Dejneka, Corning, NY (US); Joydeep Lahari, Painted Post, NY (US)

Correspondence Address: CORNING INCORPORATED, SP-TI-3-1, CORNING, NY, 14831

(21) Appl. No.: 10/787,794

(22) Filed: Feb. 26, 2004

Related U.S. Application Data
(63) Continuation-in-part of application No. 10/027,286, filed on Dec. 20, 2001.

(51) Int. Cl. C12Q 1/68; G01N 33/53; C03C 3/15; G01N 33/543

(52) U.S. Cl. 435/7.1; 436/518; 501/50

(57) ABSTRACT

Homogeneously mixed rare-earth doped particles and methods of using such particles include nano to microsized particles having a concentration of at least about 0.0005 mole percent of a Rare-Earth Oxide (Re₂O₃). The particles can be used for detecting the presence of an analyte in a sample and for detecting interactions of biomolecules.

### CROSS REFERENCE TO RELATED APPLICATION

This is a continuation-in-part of U.S. patent application Ser. No. 10/027,286 filed on Dec. 20, 2001, the content of which is relied upon and incorporated herein by reference in its entirety, and the benefit of priority under 35 U.S.C. § 120 is hereby claimed.

### FIELD OF THE INVENTION

This invention relates to detectable micro to nano sized structures or particles in general. More particularly, the present invention relates to beads and labels, methods of manufacturing detectable labels and beads and methods of using detectable labels and beads.

### BACKGROUND OF THE INVENTION

Miniaturized markers and indicators have found utility in a wide variety of areas, but they are of particular interest in biological and chemical assays. The development of multiplexing and miniaturization of chemical and biochemical assays has improved the analysis of samples in such areas as biomedical analysis, environmental science, pharmaceutical research, food and water quality control. For example, in the area of genomics, DNA arrays allow multiplexing and miniaturization of tests by providing a unique DNA target, or any other analyte, a unique address in the form of a position on the array in a small area (typically less than 100 microns in diameter) on the array surface. The total size of the surface and the spacing and size of the individual target or the analyte determines the number of addresses available.

Microtiter plates also allow multiplexing and miniaturization of samples by providing many individual wells, each at a unique position. It is possible for each well to have a unique target and to be tested with a unique sample, which allows for the multiplexing of both targets or of samples.

Another way of achieving miniaturization and multiplexing is through the use of miniaturized devices such as nanoparticles used as labels or as biological reporters for the target and microparticles used as the beads, hosts, substrates or supports attracting, targeting or otherwise selectively binding with the probe. These nanoparticles and microparticles can be provided with a unique indicia that can be identified through appropriate instrumentation such as a flow-through cell, a bead sorter, or an imaging system. Note, that in this context of a bead sorter, the bead definition can include both the nano and micro particles. Microparticles, microspheres or microbeads can be used as substrates which can be functionalized with a variety of chemical and biochemical groups, including, but not limited to nucleic acids, proteins and small molecules which act as probes for using an anti-ligand to attract a ligand. These functionalized microparticles or beads, in the larger context of labels and substrates, which actually range in size from the hundreds of microns to nanometers, can be placed in a suspension, and binding and/or interaction events can be quantified by optical techniques such as fluorescence using conventional fluorescent markers such as Cy3 and Cy5 or biological organic markers such as rare-earth chelates to fluorescently label physiologically reactive species.

The use of nanoparticles for the labels and the microparticles for the beads in the analysis of biochemical binding events offers several advantages over conventional microarrays. Since binding studies can be carried out using suspensions of particles, issues related to local probe depletion encountered with microarrays can be minimized. The use of nanoparticles and microparticles together with multiwell microtiter plates facilitates the design of highly multiplexed assays involving the binding of many different probes to many different particle types within individual wells. Although the use of nanoparticles and microparticles offers many advantages, the manufacture of such miniaturized devices has proven difficult. More specifically, the mass production of such devices in large quantities and at a low cost is particularly problematic.

One way of manufacturing these miniature devices has been developed by SurroMed, Incorporated, Mountain View, Calif., and involves making cylindrical metal nanoparticles of which the composition along the particle length can be varied in a stripe-like manner. Varying the number of stripes, the width of stripes, the identity of the metals, and the overall particle shape enables the production of a wide variety of unique labels. These “nanobarcode” tags can be identified using optical microscopy, based on the pattern of differential reflectivity of adjacent metal stripes. These identification tags facilitate multiplexing of assays in various media. However, the manufacture of these metallic nanoparticles can be technically challenging and expensive. In addition, reflectance measurements generally have a high signal to noise ratio and poor sensitivity.

A method for manufacturing microparticles is described in U.S. Pat. No. 6,268,222. U.S. Pat. No. 6,268,222 describes a core particle having on its surface smaller polymeric particles stained with different fluorescent dyes. One limitation of the use of fluorescent dyes to identify the particles is that the excitation and emission spectra of these dyes may interfere or overlap with conventional dyes such as Cy3 and Cy3 that are used for labeling reporter molecules used in bioanalysis. In addition, the fluorescence intensity of dyes tends to deteriorate over time upon prolonged or repeated exposure to light. Still another limitation of dyes is that the degradation products of these dyes are organic compounds that may interfere with biological processes and molecules being evaluated. Rare-earth chelates, which are also organic compounds, are also expensive, have low absoption cross-sections, and are not very durable.

It would be advantageous to provide miniaturized devices that could be encoded with a large number of unique identification tags and could be utilized for sensing interaction and binding of molecules. Moreover, it would be desirable if the devices could be mass produced easily and inexpensively. Furthermore, it would be useful if the devices could be identified using conventional optical detection techniques, for example, those using fluorescence detection.

### SUMMARY OF INVENTION

One aspect of the invention relates to mixed rare-earth doped particles and methods of using such particles. According to another aspect, the rare earth elements may include Ce, Pr, Nd, Sm, Eu, Gd, Tb, Dy, Ho, Er, Tm, Yb, and U and combinations thereof.

Additional advantages of the invention will be set forth in the following detailed description. It is to be understood that both the foregoing general description and the following detailed description are exemplary and are intended to provide further explanation of the invention as claimed.

### BRIEF DESCRIPTION OF THE DRAWINGS

FIG. 1 shows a schematic representation of a particle as a bead and a label according to one embodiment of the invention;

FIG. 2 shows a spectrally encoded table according to another embodiment of the invention; and

FIG. 3 is a plot of the emission spectra of a decoded particle after illumination with an ultraviolet light source.

### DETAILED DESCRIPTION

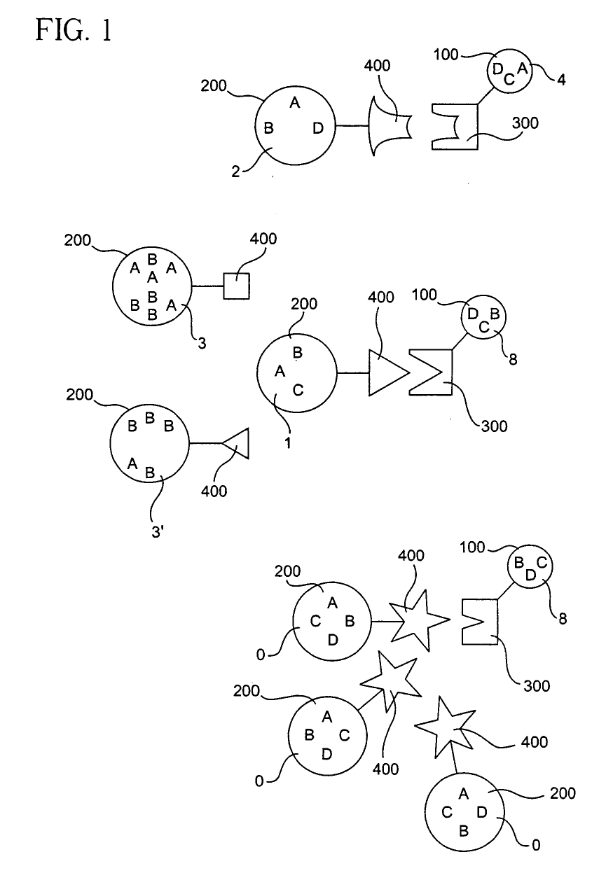

Referring to FIG. 1, the present invention relates to miniature particles 100 or 200, their manufacture and use of particles as detectable nanosized labels 100 for use as an identifier for an analyte, sample, or target 300 or as carriers, substrates, or microsized support beads 200 for attaching a unique functional group or probe 400. Even though a line is shown representing the strong binding of either the support bead 200 to the probe 400 or the nanosized label 100 to the target 300, it is to be appreciated that no actual chemical linkage needs to be formed, but rather a physical adsorption can be present to form the binding, in the form of a functional group, for example. However, the functional group can be part of the probe 400, the target 300, the support bead 200, the nanosized label 100, or the line representing their binding.

In some embodiments, the particles 100 or 200 are homogeneously doped with various combinations of rare earth (RE) elements A, B, C, or D, for example, in a glass or ceramic host. Rare earth doped glasses are preferred because of their narrow emission bands, high quantum efficiencies, noninterference with common fluorescent labels, and inertness to most organic and aqueous solvents. According to the teachings of the present invention, the particles 100 or 200 can be used as carrier beads or labels to bind with the probe or target, respectively. Thus, the particle 100 can be used as a RE target and the particle 200 can be used as a RE probe. The particle 100 can be used as a RE target with a conventional probe. Moreover, a conventional target can be used with the particle 200 as a RE probe.

The particles need not be homogeneously doped as long as the particle is not intentionally patterned for certain desired applications of bead-based-assays of biological interests. In certain embodiments, particles 100 or 200 can be manufactured by known methods of sol-gel synthesis, flame hydrolysis, spray drying, or various other techniques known to those skilled in the art. Rare earth doped particles such as microspheres and nanospheres can be made providing a coded dopant profile associated with the microsphere or nanosphere for the smaller sized nanosphere to function as a label 100 or for the microsphere to function as the bead 200. As used herein, the term label, in its most general sense, means an identifying or descriptive marker including an encoded particle which can be the label attached to the target 300 or the encoded particle used as the bead 200. To avoid the confusion between the label 100 attached to the target 300 and the general meaning of labels, the words “indicators”, “indicia”, “identifiers”, or “markers”, will be used interchangeable with labels in its more general sense of identifying anything with particles 100 or 200. The indicators and methods of the present invention are useful in fields such as chemical, biochemical, biological or biomedical analysis, process control, pollution detection and control, security, and other areas. The indicators and methods are adaptable to a wide variety of samples including biological samples and extracts (such as physiological fluids, nucleic acid and/or protein-containing solutions, microbial cultures, etc.), environmental samples (such as water sources), industrial, especially chemical reagents, products and wastes, security (such as currency, ink marking and bar-coding for authenticity) etc. According to the present invention, large numbers of particles can be simultaneously probed with a functional group 400 to determine binding and/or interaction between cells and biomolecules including, but not limited to proteins, antigens and antibodies, and nucleic acids. The particles of the present invention could be used in an unlimited number of assays such as high throughput drug screening and in vitro immunodiagnostics.

One advantage of the present invention is the ability to provide indicators used in bioanalysis that produce discrete optical signals that do not spectrally interfere with traditional fluorescent channels such as Cy3, Cy5, Texas Red, FITC, and other fluorescent reporter dyes commonly used as labels for targets. Another advantage of the present invention is that by using techniques such as sol-gel fabrication, the particles can be inexpensively and easily mass produced. The particles can contain different amounts or concentrations of rare earth elements to provide a unique identification code for each particle. Another way of providing unique identification code is to dope a particle with a plurality of rare earth elements. The elements need not be separated spatially to provide a pattern or array but can be randomly mixed similar to an abstract color painting or mixed homogeneously to form a unique fluorescent color, hue, or intensity. Each of the different rare earth elements, or same elements doped at a different concentration on the particle, as seen in bead 200 marked 3 and 3′, provides a discrete optical signal capable of detection by conventional optical equipment and can be used to identify the particle 200.

Still another advantage of the invention is that the indicators and methods of the present invention would not require new or elaborate methods of illumination or detection. Optical signals, including, but not limited to fluorescent signals can be collected by an imaging detector such as a charge coupled device (CCD) camera and image analysis can be utilized to determine quantitative assay data. Other systems that could be used for detection for use with the indicators and methods of the present invention include an excitation source, a light filter and a detector array.

The particles of the present invention may be attached or associated with a specific binding molecule or other functional groups 400 so that the particles 100 or 200 can be utilized in the detection of biological or chemical compounds and interactions of biomolecules with other biomolecules or chemicals. Attachment or association of the particles 100 or 200 of the present invention to chemicals or biomolecules can be accomplished using techniques known in the art. An advantage of certain embodiments of the present invention is that in embodiments in which the particles 100 or 200 are made from glasses or ceramics (especially silicates), attachment of biomolecules and functionalization of such surfaces with coatings or layers with chemicals to facilitate attachment of biomolecules is well-known. For example, coating of silicate glass surfaces such as high density microarray and microwell surfaces to promote binding or attachment of biomolecules is known in the art. The surfaces of inorganic substrates can be modified by the deposition of a polymeric monolayer coating or film to construct biomolecular assemblies. In addition, surface modification can also be used to promote adhesion and lubrication, modify the electrical and optical properties of the substrate surface, and create electroactive films suitable for various optical and electronic sensors and devices. Compounds with amine functionality have found extensive application in the preparation of surfaces for nucleic acid hybridization. Due to their ability to bond to a substrate with a hydroxide and their ability to bond to nucleic acids with an amine, silane compounds are useful as surface coatings that will effectively immobilize nucleic acids. One example of a silane used for biological assay preparation is gamma amino propyl silane (GAPS), which may be deposited by a variety of methods, including CVD, spin coating, spray coating and dip coating. It will be understood that the particles 100 or 200 used in accordance with the present invention can be functionalized with virtually any surface chemistry compatible with the particle surface, and the invention is not limited to a particular surface chemistry. In embodiments in which the particles are made from alumino-sillicate glass, the particles are extremely durable in organic solvents such as ethanol, isopropanol, chloroform, dimethylsulfoxide, dimethylformamide and hexane. Glass particles do not swell or dissolve in such solvents, unlike polystyrene encoded spheres.

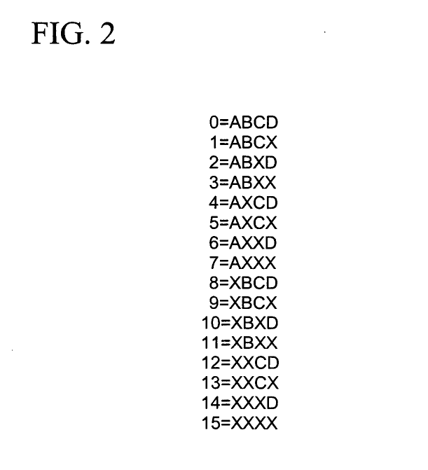

According to one embodiment of the invention, the glass particles are encoded to provide a unique identification code 0, 1, 2, 3, 3′, 4, and 8 for example in FIG. 1 for a sufficient or medium number of individual particles for use in biological assays. Preferably, according to the present invention, more than 100 and less than about 1000 unique codes can be provided. The present invention also provides a relatively simple process to mass produce such particles from inexpensive and readily available materials, processes and equipment. Methods that are already known can be used to make the inventive glass particles via sol-gel or other existing methods. Glasses can be made, according to Table I or II, and doped with various rare earth (RE) ions A, B, C, or D, in any combination as illustrated in FIG. 1 and FIG. 2. The glass components would be batched and mixed together and then heated in a Pt, SiO₂ or other refractory crucible to form a melt. The glasses were then crushed and spherodized to make 5-50 micron sized spheres for use as beads 200. For the smaller application using nanosized spheres, for used as labels 100 for targets 300, the nanosized particles 100 could be made via the well-known sol-gel method or chemical vapor condensation method. The labeling of biological molecules or other targets 300 can be readily accomplished using well established silane chemistries. For example, RE labeled cDNA could be obtained by incorporating aminoallyl containing dNTPs during reverse transcription reaction followed by the addition of RE nanoparticles derivatized with silanes presenting terminal N-hydroxysuccinimidyl esters. Hence, in general RE glasss or ceramic particles are useful relative to conventional organic dyes or RE chelates because the particles can be made highly specific through appropriate chemistry (e.g. use of mixed silanes, one containing a functional group for attachment to the analyte and the other providing non-binding character (e.g. oligo(ethylene glycol)-silane) so as to minimize non-specific binding.

For biological assay applications that require inexpensive indicators but not necessarily a lot of different indicators, such as the 5 different types of blood types, what could be the most useful is a homogenous particle.

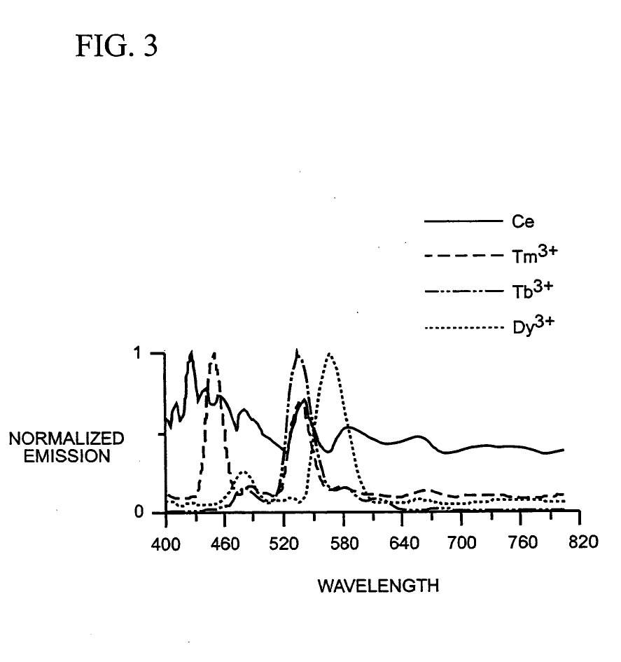

For homogenous unpatterned addressing with small microparticles for use as support beads 200, for example, there will be a need to color mix to obtain lots of combinations. By mixing together dopants that fluoresce with different colors, various fluorescent colors, hues, and intensities can be obtained for each combination. Homogeneous, in the context of the present invention, means the particles are at least by design have their dopants intermixed in a way that the color emissions are not spatially separated out in that bead. The dopants may not be actually micro-or-nano-homogeneous but essentially only one intermixed color is detected by the naked eye, such as a purple from a red and blue mix, instead of detecting a red portion next to a blue portion. However, as these red and blue portions grow smaller and smaller to become near-nano-homogenous, a pseudo-purple homogenous interior results along with the homogenous purple-like surface. To the human eye the homogenous particle will look like a mixed color, but a spectrometer or set of filters will be able to see the red and blue individually and distinguish their individual intensities, as decodable by FIG. 3. The naked eye can also distinguish the relative intensities by the shade of the color and the total brightness. The dopants are thus mixed (red and blue together to form purple) all embedded together in an inexpensive inorganic glassy or crystalline host, homogenously. The homogenous bead can then be spectrally resolved by looking at the amount of yellow versus blue or green or whatever the individual color dopants are.

For example, the red fluorescing dopant and the green fluorescing dopant are mixed in some proportion or ratio in the same batch to make a glass that will fluoresce various shades of yellow and orange fluorescence depending on the proportions of red and green fluorescing dopants. The glass can be formed into spherical particles by a number of very well known and inexpensive processes.

These uniquely coded glass particles can be used in a wide variety of sensing and indicating applications. For example, after preparation of the glass particles 100 or 200, which have a unique identification code 0, 1, 2, 3, 3′, 4, and 8 for example in FIG. 1 based on the optical properties, color and color intensity due to the concentration of the dopants, the glass particles can be placed in contact with a chemical functional group 400 or a target 300 for further analysis. For example, each glass particle could be associated, as a bead 200 with a code 0 or a label 100 with the code 8 for the target 300, with the target being an analyte such as an antibody, a target DNA, a pharmaceutically active compound, a drug compound, etc, or any other target, analyte, or sample. The particles associated with various analytes could then be used in a wide variety of assay formats, for example, where the binding and/or interaction of one or more molecules are being measured through the tagging of one of the reactants 400 with a unique code, such as a color encoded bead 200 marked 0, and the other reactant with a simple indicator, such as a label 100 marked 8 for the target 300. Attachment and binding of biomolecules may be facilitated by providing an appropriate surface chemistry on a surface of the particles that could serve as either the label for the target or the encoded bead, depending on the size of the particle.

Unpatterned but color encoded, “homogeneous” beads are shown in FIG. 1. These homogeneous beads fluoresce in many different colors. For example, florescent red, green, or blue, as an initial color or primary color spectrum for use as a base for the precise encoding of rare-earth doped glass nanoparticles or microparticles with shades by varying the dopant concentration.

Rare-earth doped glass nanoparticles are little particles or colloids that fluoresce. As is known, one μm is 1000 nanometers. Once the particles are reduced 1000 times smaller than the microparticles, then one can use the nanoparticles 100 to piggyback them as a label for a biological cell or some other kind of target 300. By using color combinations, such as by varying the relative concentrations of the different dopants, more codes such as the combinations marked 4 and 8 can be formed.

As the carrier or support, the size of the glass bead can be as small as about 5 microns or smaller for a spherical glass beads. Furthermore, for larger size particles, one can use patterned glass beads, and for smaller particles, one could just use homogeneous beads 200. Small color-coded beads 200 can be used to study the binding of the functional group 400 to a target 300, or vice a versa. Traditionally when looking at the binding of the target 300 to the functional group 400, it is implied that the target 300 is going to come in and bind to the functional group 400. The functional group 400 is already there in a vessel or another container for example. Under those circumstances, organic dyes are typically attached to the target 300 as labels. As is known, these organic dyes have low background noise and have other special properties. Basically organic fluorescent dyes satisfy these criteria. Then what one is studying is not the binding of the target 300, but how much of the dye is detected and from that, one can infer how much of the target 300 has bound to the functional group 400. Labels and reporters are interchangeably used but specifically, reporters are biological labels. Biological labels can be a protein or it could be a read out of some path way downstream, but basically it is used in a biological sense for labeling.

Biological labels are nanometer sized entities because larger sized micrometer particles would be too big and not work very well as a label to be able to study any reasonable binding. The biological material such as a cell would spit-out the larger micrometer brick-like particle. The primary forces would be the sedimentation of one these label particles. Hence it is desired that the labels are smaller than 100 nanometers. Pushing it beyond a couple of hundred nanometers and the label may not stick. Thus, a one hundred micrometer bead particle can never be a label or at least not an efficient one for biological assays. Hence, labels should be significantly less than a micrometer, it could probably be 200 nm, but that is almost the limit. The maximum size would be 200 or 300 nm but not much larger than that.

Beads are essentially small particles in the μm range but serve a different function than labels. For the types of beads of interest, they are in micrometers, 1000× bigger than the desired label. The bead 200 is attached to the functional group 400 and one is going to study the binding of the target 300 to the bead attached functional group 400 using fluorescent labels that are conventionally fluorescent organic molecules, i.e. a dye molecule. The dye is just added color but a Rare Earth chelate is an example of a dye molecule.

Many different kinds of beads in the micrometer range are used as the host for studying the binding reaction. The micrometer sized beads are formed with different rare earth dopants to form beads of many colors. For example, assume a triangular functional group 400 was attached to a red colored bead 200 marked 1 and a blue colored bead 200 marked 0 was attached to a star-shaped functional group 400, then when the binding of the star shaped functional group 400 was studied, one is actually studying the binding of the target 300 labeled 8 to the triangular functional group marked 0 with the bead 200. In the event one saw lots of binding of the fluorescent dye label, which could also be a rare-earth doped label 100, but more typically, it is an organic dye, one would say the entity that was immobilized on the blue fluorescing bead 200 marked 0 had a tighter infinity for the target 300 labeled 8. Sine the beads 200 were prepared in such a way that pre-attach the star-shaped functional groups 400 to the blue beads 200 marked 0, one can then say that these beads 200 marked 0 have a higher affinity for the target labeled 8. Hence, the bead 200 marked 0 acts as an address for different types of bio-molecules or other functional groups 400. A beaker or a test tube could contain red fluorescing beads or blue fluorescing beads for studying the binding of the target 300 labeled 8 for the two different beads 200 marked 0 or 1, amongst other possible beads 200 marked 2, 3, or 3′. If the target 300 labeled 8 has yellow fluorescence and if one sees greater yellow fluorescence coming out of the blue fluorescing particles 200 marked 0, that inference can be made. Now imagine extrapolating that to 100, 1000, 10000 or whatever the different rare-earths allow and the possible combinations of the rare-earths allow one to make 1000s and 1000s of different addresses for studying binding attractions. This is what is meant by the site for where the binding assay is. By having a different color which is a unique identity in many ways, the homogeneous encoded particle acts like a bar code.

In accordance with the teachings of the present invention, the rare-earth doped glass particles can be used as the beads 200 or labels 100 for the target 300. The beads 200 serve as the site of the reaction and a unique identifier for the binding reaction sites. As labels 100, the rare-earth doped nano-glass can be produced inexpensively in large numbers.

In another aspect of the invention, a glass particle can be prepared that has an optical property that is different from an optical property of another particle. The various different dopants A, B, C, or D, for example, provide a unique code as seen in the table of FIG. 2, for the different fluorescent material to provide an unpatterned fluorescent variation. In another aspect, the concentration of each of the dopants A, B, C, and D can also be varied, as seen in the beads 200 marked 3 and 3′, to increase the number of variables available to provide a unique identifier for each glass particle. Additionally, the size of each glass particle can vary, as seen in the beads 200 marked 3 and 3′ to further increase the number of variables available to provide a unique identifier. The small size preferred for biological assays is about 5, 10, or 20 μm for the spherical microspheres as used as beads 200.

For each rare-earth dopant, different levels of intensity can be used for Boolean multiplexing. The rare earth is used for the address for homogeneous spherical bead bio-assay. Hence, if one knows the size (preferably two different sizes) of the particle and intensity one can multiplex to a greater degree. However, the size should not vary too much because the hydrodynamics dictate what size works best. Generally, for the maximum interflow between the particles, similar sized particles flow better.

A color encoded glass particle can thus be used as a detectable label 100 for attachment to the target 300 or as a bead support 200. The glass particles can be detected by a scanner or a suitable detector. For example, glasses that fluoresce red, green and blue can be assembled together to provide emissions of varying optical properties. For example, dopants that fluoresce red, blue and green can be mixed together to form a new shade (red+green=yellow or orange, red+blue=purple) homogenously on the particle, allowing the dopants to disperse in a random manner. Tiny portions of red, blue, and green will appear intermixed to the naked eye but can be detected with a spectrometer or using a set of filters as individual red, blue, and green peaks at their specific intensity level. The relative ratios of the peak heights of the different fluorescence bands are detected. For example in FIG. 3, the absolute or relative intensities of the emission bands at 425 (Ce), 455 (Tm³⁺), 542 (Tb³⁺), and 575 nm (Dy³⁺) can each be varied independently within the dynamic range of the detection system used to create thousands of different and unique indicia.

Up to 13 different RE ions selected from the group comprising Ce, Pr, Nd, Sm, Eu, Gd, Tb, Dy, Ho, Er, Tm, Yb, and U could be doped in various combinations and proportions in a nanoparticle 100 or microparticle 200 to obtain many unique combinations. In general, RE doped glass particles, and specifically nanoparticles 100 have much better durability than organic tags and RE chelates. RE doped glass particles also can be used for time gated fluorescence since their lifetimes are even longer than the chelates to provide outstanding signal to noise. The RE doped glass particles are not photosensitive like most organic tags making them easier to handle. Typically only a few RE chelates can be bound to a strand of DNA or protein. Likewise only a few RE nanoparticles could be bound to the same strand. However each nanoparticle 100 could contain up to 50 RE ions (given 10 nm spheres, with 1×10\hat{ }20 RE ions/cc) giving a 50 times improvement in fluorescence signal. This is the maximum concentration that can be used before concentration quenching begins to limit the fluorescence intensity of Ce, Pr, Sm, Dy, Ho, Er, Tm, and U. However, for Eu and Tb (and Yb in the IR), which do not suffer from concentration quenching, the concentration can be increased 10 times higher before the intensity becomes nonlinear so the ultimate fluorescent signal could be a factor of 500 greater than RE chelates. Hence, Tb, Eu, (and Yb in the IR) are ideal candidates for used as the nanosized “labels” for the targets. It is to be appreciated that if the RE particles are used as the nanosized labels, interference with the substituted organic dye labels is an irrelevant issue.

However, if potential interference with organic dye labels is a relevant issue, then the preferred set of dopants for use as the microsized beads 200 are Ce, Tm, Tb, and Dy.

Since inorganic hosts have lower phonon energies than chelates, many more RE transitions are accessible giving rise to a larger number of possible dyes for multiplexing. Thus, not only could visible fluorescence be used, but also near IR emittors like Yb, Nd, Er and Tm could be used and at wavelengths where tissues are nonabsorbing enabling in vitro studies and diagnostics. These NIR transitions are typically quenched by the high phonon energy of the chelate host. The narrow intra f transitions of the RE ions are very narrow which also allows for highly multiplexed labeling and as many as 9 visible (Ce, Pr, Sm, Eu, Tb, Dy, Ho Tm, and U), 2 ultraviolet (Gd and Tm), and 4 near IR labels (Yb, Nd, Er and Tm) could be used. In addition the RE's can be mixed and intensity multiplexed to provide even more unique labels for targets if necessary. Finally the RE's provide yet another method of detection via upconversion. Ions such as Pr, Ho, Er, and Tm can be pumped in the NIR and yield visible fluorescence without any background at all from the biological material under study. In addition to assays this technique can also be used for high resolution imaging of structures within tissue below the diffraction limit of the pump due to the nonlinearity of the upconversion pump. This effect can also be enhanced with appropriate codopants such as Yb.

It is also possible to rapidly synthesize many different unique indicia. Unencoded silica spheres could be placed into the 10 μL wells of a 1536 plate (Corning, Inc.). The identification would be applied by solution doping, sol-gel, CVD, or other suitable process. Different amounts of the selected dopants used could be automatically dispensed into each of the wells to build the codes.

In another embodiment of the invention, a method of detecting multiple analytes or samples is provided by using spectral imaging. Spectral imaging is a technique in which the spectral properties of every point or pixel in an image are captured. For example, when many fluorescent coded microparticles are excited and in the field of view, the emission spectra (as a function of wavelength) of every pixel in the image is captured. Thus non overlapping peaks can be easily resolved and simple computer algorithms can be used to decode or interpret the identities of all the coded particles in the field of view. Using spectral imaging, the spectra at each point can be acquired, and the composite image obtained directly in a single scan via a computer algorithm. When combined with the coded particles of the present invention that can all be excited with the same light source, the resulting spectral image can be used to decode all of the coded particles with one image. Thus spectral imaging can simplify measurements and increase throughput as well as enable unique analysis of assays in which multiple tags or coded particles are present.

With spectral imaging experiment, coded particles can be decoded with a UV lamp and a spectral imaging microscope. For example, the coded glass particle can include a first concentration of Tm and Dy, a doubled concentration of Tb and a ½ concentration of Ce. A 100 W 365 nm Hg lamp can be used to illuminate the particle and the fluorescence signal from each of the four different RE ions in the particle can be easily resolved. Even more importantly, the four resultant colors from the four dopants and increased intensity from the two doubled dopants, of the coded particle would also be spectrally resolvable. FIG. 3 shows the fluorescence spectrum of a particle in which the emission spectra were used to decode the fluorescent signature of the particle. Hence, according to the present invention, coded particles can be decoded using a simple computer algorithm and an inexpensive UV lamp.

It will be understood, of course that this relatively simple example is exemplary of the present invention, and more elaborate spectral imaging systems can be designed by those of skill in the art. For example, using a microscope with a built-in, focused UV light source should be sufficient to decode smaller particles. Alternatively, the absorption of the glass particle can be increased by increasing the dopant levels (at the sacrifice of quantum efficiency) or adding a sensitizer like Ce to absorb the UV and transfer it to the fluorescent ion.

Without intending to limit the invention in any manner, the present invention will be more fully described by the following example.

### EXAMPLES

Tables I and II below show exemplary glass compositions that can be used to make a fluorescent glass particle in accordance with one embodiment of the invention. The glass compositions in Table I and Table II were chosen because fluorescent rare earth materials are soluble in these glasses. It will be understood, however, that the present invention is not limited to a particular glass composition.

TABLE I

Composition (Weight %)

| | 1 | 2 | 3 | 4 |
|---|---|---|---|---|
| SiO₂ | 50.61 | 51.22 | 52.65 | 52.40 |
| Al₂O₃ | 14.56 | 14.73 | 15.14 | 15.07 |
| B₂O₃ | 7.39 | 7.49 | 7.70 | 7.66 |
| MgO | 0.64 | 0.65 | 0.67 | 0.67 |
| CaO | 3.65 | 3.70 | 3.79 | 3.78 |
| SrO | 1.67 | 1.69 | 1.73 | 1.72 |
| BaO | 8.24 | 8.34 | 8.57 | 8.54 |
| Eu₂O₃ | 6.58 | 0 | 0 | 0 |
| Tb₂0₃ | 0 | 6.93 | 0 | 0 |
| CeO₂ | 0 | 0 | 0.23 | 0 |
| Tm₂O₃ | 0 | 0 | 0 | 0.99 |
| Y₂O₃ | 0 | 0 | 4.11 | 3.79 |
| Sb₂O₃ | 1.45 | 0 | 0 | 0 |
| F | 0.24 | 0.24 | 0.24 | 0.24 |

TABLE II

Composition (Weight %)

| | 5 | 6 | 7 | 8 |
|---|---|---|---|---|
| SiO₂ | 55.46 | 55.31 | 59.53 | 59.69 |
| Al₂O₃ | 0.96 | 0.95 | 1.03 | 1.03 |
| Li₂O | 1.60 | 1.60 | 1.72 | 1.72 |
| Na₂O | 3.32 | 3.31 | 3.56 | 3.57 |
| K₂O | 5.04 | 5.03 | 5.41 | 5.43 |
| SrO | 9.70 | 9.67 | 10.41 | 10.44 |
| BaO | 10.25 | 10.23 | 11.01 | 11.03 |
| ZnO | 6.54 | 6.52 | 7.01 | 7.03 |
| Eu₂O₃ | 7.06 | 0 | 0 | 0 |
| Tb₂O₃ | 0 | 7.32 | 0 | 0 |
| CeO₂ | 0 | 0 | 0.25 | 0 |
| CaO | 0.01 | 0.01 | 0.01 | 0.01 |

In each of the encoded glass particles, the glass particle doped with Eu₂O₃ produced red fluorescence, the glass particle doped with Tb₂O₃ fluoresced green, and the glass particle doped with Tm₂O₃ and CeO₂ fluoresced dark or sienna blue and powder blue, respectively, while the undoped particle showed no fluorescence. These dopants are exemplary only, and a wide variety of other dopants could be used in accordance with the present invention. In addition, mixtures of dopants could be used to produce a wider variety of fluorescent colors, such as the red fluorescence from Eu₂O₃ added to the blue from either Tm₂O₃ or CeO₂ to produce dark or light purple, respectively. The glasses produced in this Example can be excited with a mercury lamp at either 254 or 365 nm. At these wavelengths, fluorescent tags or labels for the targets or analytes such as the commonly used Cy-3 and Cy-5 for the DNA target or analyte are not excited, and therefore, crosstalk and interference between identifiers in the glass particles and the DNA labels is not an issue.

Any suitable detection system can be utilized to detect the difference in optical properties among the color encoded particles of the present invention. For example, in a simple system, a microscope could be utilized. Other systems could utilize a solid state detector, a photomultiplier tube, photographic film, or a CCD device used together with a microscope, a spectrometer, a luminometer microscope, a fluorescence scanner, or a flow cytometer. A wide variety of optical properties can be varied among the particles according to the present invention. Such optical properties include, but are not limited to difference in fluorescence lifetime, difference in fluorescent intensity, difference in wavelength of emission, difference of fluorescent polarization, and combinations of these properties. The invention is widely adaptable to a variety of sensing applications, including, but not limited to, clinical, forensics, genetic analysis, biomolecular analysis and drug-discovery efforts.

According to the teachings of the present invention, rare-earth doped homogeneously mixed particles are taught. Such rare-earth doped particles are especially useful as microbeads for multiplexing bio-assays. Bio-assays is a novel field of use for rare-earth doped particles. In bio-assays, lower numbers of microbeads (such as less than 100 or at most 1000 is sufficient. However, low cost and small size (microparticle) are both paramount.

Color-encoded particles are doped with at least one rare-earth element where that rare-earth has a unique identifier for use as an address for the bio-assay. The use of RE-doped beads in other types of applications is not obvious just because there is quite a bit of work involved. In order to use such particles, it is needed to find the bounds or numbers of particles or numbers of ions to maximize the use of these small doped microparticles for multiplexed bio-assays.

Not all rare-earths will work for all applications. Distinct elements that do not interfere with organic dyes can be chosen such as Ce, Tb, Dy, and Tm if compatibility with organic labels such as Cy-3 and Cy-5 is desired.

Specifically, the minimum concentration of RE ions required for sufficient detection of the beads has to be determined. Other questions include what is the maximum rare earth concentration that can be achieved before concentration quenching sets in and the fluorescence intensity no longer increases linearly as a function of concentration, at what fluorescence intensity does the detection system become saturated and therefore how many grey levels of intensity is possible for at least a few of the RE ions for a homogeneously microbead to be intensity resolved.

The minimum concentration of detectable rare earth dopants will be determined by the brightness of the excitation source, the efficiency of the fluorescent ions, the size of the spheres, the water content of the glass, the speed at which the fluorescent spheres or beads need to be read, the numerical aperture (NA) of the collection optics, and the sensitivity of the detection system, and other factors, but assuming reasonable numbers, a rough minimum will be about 400 ppm or 0.01 mole % (as Re2O3) with a 500 W Hg lamp for 4-5 micron sized microspheres. A laser source, instead of the Hg lamp, would drop the minimum concentration down to about 40 ppm or 0.001 mole % (or better). There is some efficiency variance among the rare earths but only by a factor of 2 or so, and again depends largely on the excitation wavelengths used.

The upper limit for doping of Ce, Pr, Sm, Dy, Ho, Er, and Tm is about 0.25 mole % or 10000 ppm, before concentration quenching sets in and the fluorescence intensity ceases to linearly increase with concentration. As is known, quenching is the nonradiative (no light emitted) de-excitation of an excited ion, usually due to nonradiative energy transfer between the ions. For example if 0.01 mole % Tm₂O₃ normally gives a fluorescence intensity of 1 unit, then 0.25% Tm₂O₃ gives 25 units. However when the Tm₂O₃ is increased to 0.5% only 26 units of fluorescent intensity is observed indicating the onset of concentration quenching is between 0.25 and 0.5 mole % Tm₂O₃. Like wise other rare earth ions can quench each other. When 1% Tb₂O₃ is added to a 0.25% Tm₂O₃ glass, its Tm³⁺ fluorescence intensity typically drops by an order of magnitude, where as 1% Tb₂O₃ can be added to a Eu₂O₃ doped glass without any decrease in Eu³⁺ luminescence. So if the upper concentration is typically bound by the onset of concentration quenching and the lower limit by the sensitivity of the detection optics. For Eu and Tb (and Yb in the IR), the single dopant concentrations can be increased to about 10 mole % before quenching occurs.

Since there are errors in determining the exact intensity of a fluorescence band, making adjacent grey scale levels vary by a factor of 2 leaves ample room for accurate assignment. The minimum concentration limit for Ce, Pr, Sm, Dy, Ho, Er, and Tm will be determined by the detection system sensitivity and limited to about 400 ppm or 0.01 mole % (as Re2O3) with a 500 W Hg lamp while the upper is determined by quenching at about 0.25 mole % or 10000 ppm. Thus there are 5 grey scale levels that are easily measurable between 400 and 10,000 ppm of dopant. For Eu and Tb (and Yb in the IR) where much higher dopant levels are possible 9 grey levels can be obtained between the upper and lower dopant limits.

Thus a conservative estimate of the possible number of combinations in which compatibility with dye labels is required is 4 colors raised to 5 grey levels would give 1024 combinations. All of these combinations may not be possible since all the dopants all at their high concentration will quench each other, so about 1000 practical combinations are possible.

New violet and ultraviolet lasers which are becoming available have far greater power than Hg lamps and will greatly reduce the minimum detectable level of doping by a factor of 10. With such lasers, the minimum detection limit would be reduced to 0.001 mole % for Re₂O₃. However, a concentration of 0.005 mole % is preferable for a better signal to noise ratio. These lasers would allow for 3 more grey levels and increase the possible combinations to 5 colors raised to 8 grey levels resulting in 390,625 or more combinations. Combinations can be further expanded by taking advantage of the extended concentration limits of Eu and Tb. However, Eu and Tb may swamp the other colors when the 10 or 20 mole % levels are used and the detection system will most likely not have the dynamic range to see the intensity, unless the excitation energy can be changed on the fly to increase the dynamic range.

There are many assumptions used in the approximate numbers used as estimates. For example, if the size of the beads or particles varies by 50%, then so will the fluorescence intensity. Hence, maybe more than a factor of 2 is needed to distinguish grey scales.

Since there will be some error around assigning a particular grey level to an intensity, some error tolerance have to be built in. In the digital world it is easy and the intensity is either 0 or 1. A factor of 2 is used in the following example to accommodate for possible tolerances. Hence, if the maximum intensity is 100% then with a factor of 2 increment, the grey levels would be graded as follows, with examples of concentrations in the Table III.

TABLE III

| | |
|---|---|
| % | Mole % Re₂O₃ |

| Relative | Ce, Pr, Sm, Dy, | Eu or Tb |
|---|---|---|
| Intensity | Ho, Er, and Tm | (or Yb in the IR) |
| 6400 | Concentration Quenched | 16 |
| 3200 | ″ | 8 |
| 1600 | ″ | 4 |
| 800 | ″ | 2 |
| 400 | ″ | 1 |
| 200 | ″ | .5 |
| 100 | 0.25 | 0.25 |
| | (without Eu or Tb) | |
| 50 | 0.125 | 0.125 |
| 25 | 0.0625 | 0.0625 |
| 12.5 | 0.03125 | 0.03125 |
| 6.25 | 0.015625 | 0.015625 |
| 3.125 | 0.0078125 | 0.0078125 |

This grey scale makes it easy to distinguish the different intensity levels. It is too be appreciated that when a large number grey levels is used, it is hard to see the low levels if there is a high one in the same place like a bead with 100 Green and 3.125 Red. The 3.125 red may get washed out by the overwhelming intensity from the 100 Green. It may also be possible to create many more possibilities by going to smaller differences in gray scale levels like factors of 1.5 or 1.2 if tight reproducible beads are made and the detection optics can reliably distinguish 20% differences in intensity. A non-uniformly graded grey scale is also possible as 20% differences may be easy to distinguish in the brighter, highly doped glasses, while 50% differences may be required from the dimmer low concentration glasses.

The preferred ratios of ions for each possible combination would be a daunting task to confirm for all 390,625 combinations and will be very dependent on the actual optical detection system used. If variable attenuation or excitation of the signal is not used, then the dynamic range of the detection system (probably 3 orders of magnitude at best) will probably set the upper and lower limits, rather than the RE concentration which can vary from 0.001, to 10 mole % (4 orders of magnitude), and even up to 20 mole %.

As one possible combination, all dopants of Ce, Pr, Nd, Sm, Eu, Gd, Tb, Dy, Ho, Er, Tm, Yb, and U can be used altogether for an extreme example if all the concentrations are on the low side (<0.1 mole %) and visible as well as IR detection is used. This will also cause interference with Cy-3 and Cy-5 and other organic dyes if they are used to label. There will also be significant spectral overlap of some of the RE emission bands. This is not a practical example but an extreme illustration of what may be possible.

Note the unique combinations of Table IV as a simpler example:

TABLE IV

| | | Fluorescence Peak Heights (% Relative) | |
|---|---|---|---|
| Dopant Concentration (mole %) | | 542 nm | 612 nm |
| Tb | Eu | (green) | (red) |
| 0.25 | 0 | 100 | 0 |
| 0.125 | 0 | 50 | 0 |
| 0 | 0.125 | 0 | 50 |
| 0.125 | 0.125 | 50 | 50 |
| 0.0625 | 0.0625 | 25 | 25 |
| 0.125 | 0.0625 | 50 | 25 |

Many other combinations are possible by mixing 2 or more of any of the dopants disclosed. The number of unique possibilities is given by C^{N}, where C is the number of dopants or spectrally resolvable colors and N is the number of grey levels.

Table V show exemplary glass batch compositions of two low concentration examples in weight and mole percentages that can also be used to make fluorescent glass particles. Note that the left set of numbers is batch weights in weight %, and the right set is in mole %.

TABLE V

| Composition | (Weight %) | (Mol %) | (Weight %) | (Mol %) |
|---|---|---|---|---|
| SiO₂ | 59.36 | 69 | 58.84 | 69 |
| Al₂O₃ | 1.02 | 0.7 | 1.01 | 0.7 |
| Li₂O | 1.71 | 4 | 1.70 | 4 |
| Na₂O | 3.55 | 4 | 3.52 | 4 |
| K₂O | 5.40 | 4 | 5.35 | 4 |
| SrO | 10.38 | 7 | 10.29 | 7 |
| BaO | 10.97 | 5 | 10.88 | 5 |
| ZnO | 6.99 | 6 | 6.93 | 6 |
| Eu₂O₃ | 0.00 | 0 | 0.05 | 0.01 |
| Tm₂O₃ | 0.55 | 0.1 | 1.37 | 0.25 |
| CaO | 0.01 | 0 | 0.01 | 0 |

A batch composition with a low concentration of Cerium oxide could also be realized with 0.05 mol % of CeO₂ as other possible combinations.

It will be apparent to those skilled in the art that various modifications and variations can be made to the present invention without departing from the spirit or scope of the invention. Thus, it is intended that the present invention cover modifications and variations of this invention provided they come within the scope of the appended claims and their equivalents.

What is claimed is:

1. A biological-library indicia glass or ceramic composition comprising a micro to nanosized particle homogeneously doped with at least two different rare earth (RE) elements.

2. The composition of claim 1, wherein the rare earth element is selected from the group consisting of Ce, Pr, Nd, Sm, Eu, Gd, Tb, Dy, Ho, Er, Tm, Yb, U, and combinations thereof with the total rare earth component varying between 0.001 and 20 mole %.

3. The composition of claim 2, wherein the particle includes a plurality of rare earth elements composed of various mixtures thereof having a composite spectral fluorescence signature for providing a unique identification code for the particle.

4. The composition of claim 3, wherein the particle is nanosized and includes at least three rare earth elements to form a label on an analyte for binding with a microsized bead that is adapted to detect the analyte or interaction of two molecules.

5. The composition of claim 3, wherein the particle is microsized and includes at least two rare earth elements to form a bead adapted to detect the affinity of a labeled molecule in an analyte to the bead or interaction of two molecules.

6. The composition of claim 3, wherein the rare earth element is in a concentration of at least about 0.05 mole percent of a Rare-Earth Oxide (Re₂O₃) where Re is selected from Ce, Pr, Sm, Eu, Tb, Dy, or Tm.

7. The composition of claim 3, wherein the particle includes materials selected from the group consisting of inorganic materials, silicates, glasses, alumino-silicate glasses, and combinations thereof.

8. The composition of claim 3, wherein the rare earth element is dispersed in a random manner.

9. The composition of claim 3, wherein the size of the particle is also used as an indicia.

10. The composition of claim 1, wherein the rare earth element is selected from the group consisting of Ce, Tb, Dy, Tm, and combinations thereof to form the microsized particle in the presence of organic dye labels.

11. The composition of 3, wherein the rare earth element is in a concentration below the onset of concentration quenching of less than about 0.25 mole percent of a Rare-Earth Oxide (Re₂O₃) wherein the Rare-Earth component is selected from the group consisting of Ce, Pr, Nd, Sm, Dy, Ho, Er, Tm, or U and combinations thereof.

12. The composition of claim 7, wherein the particle includes a chemical or biological functional group bound thereto for interaction with an analyte or biomolecule.

13. The composition of claim 12, wherein the particle includes a surface treatment to facilitate binding of biomolecules thereto.

14. The composition of claim 1, wherein the particle includes a surface treatment to facilitate binding of biomolecules thereto.

15. The composition of claim 12, wherein the chemical functional group is selected from the group consisting of a nucleic acid, an antibody, a protein, and an enzyme.

16. The composition of claim 1, wherein the rare earth element is selected from the group consisting of Eu, Tb, Yb, and combinations thereof to form the nanosized particle having maximum brightness.

17. The composition of claim of 3, wherein the rare earth element is in a concentration of less than about 20 mole percent of a Rare-Earth Oxide (Re₂O₃) with Re selected from Eu, Tb, or Yb.

18. The label of claim 1, wherein the particle has a cross-sectional dimension of less than about 20 micrometer.

19. A method of detecting multiple functional groups comprising the steps of:
providing spectrally coded glass particles, each of the glass particles having a functional group associated therewith;
illuminating the glass particles with a light source;
obtaining a spectral signature of the glass particles, wherein the spectral signature of each individual particle includes the fluorescent emission from at least two different rare earth elements; and
utilizing the spectral signature to decode the glass particles, wherein the rare earth elements are randomized to provide a unique code for each glass particle based on the fluorescent emission from at least one rare earth element.

20. A biological-library indicia composition comprising:
a plurality of coded rare-earth homogeneously doped nano to micro sized glass carriers, each having N>1 specified rare earth dopants and one of M>1 detectable intensity levels for each color, such that each carrier can be identified by one of up to M to the N power of different code combinations; and
a different known biological compound carried on each different-combination carrier.
